(19) United States
(12) Patent Application Publication
(10) Pub. No.: US 2025/0228785 A1
(43) Pub. Date: Jul. 17, 2025

(54) MICROCAPSULE, PREPARATION METHOD THEREFOR, AND USE THEREOF IN PREVENTING AND/OR TREATING DAMAGE TO SALIVARY GLANDS CAUSED BY RADIATION THERAPY

(71) Applicants: CAPITAL MEDICAL UNIVERSITY, Beijing (CN); BEIJING STOMATOLOGICAL HOSPITAL, CAPITAL MEDICAL UNIVERSITY, Beijing (CN)

(72) Inventors: Songling WANG, Beijing (CN); Yuji WANG, Beijing (CN); Wenbin LI, Beijing (CN); Xiaoyu FENG, Beijing (CN); Wen PAN, Beijing (CN); Zhifang WU, Beijing (CN); Jian ZHOU, Beijing (CN)

(73) Assignees: CAPITAL MEDICAL UNIVERSITY, Beijing (CN); BEIJING STOMATOLOGICAL HOSPITAL, CAPITAL MEDICAL UNIVERSITY, Beijing (CN)

(21) Appl. No.: 18/685,165

(22) PCT Filed: Mar. 25, 2022
(86) PCT No.: PCT/CN2022/083010

§ 371 Date: Feb. 20, 2024

(30) Foreign Application Priority Data
Aug. 19, 2021 (CN) 202110951722.5

(51) Int. Cl. A61K 9/50; A61K 9/00; A61K 31/375; A61K 33/00

(52) U.S. Cl. CPC A61K 9/5047; A61K 9/0053; A61K 9/5036; A61K 9/5089; A61K 31/375; A61K 33/00

(57) ABSTRACT

A microcapsule containing vitamin C and nitrate, said capsule comprising a wall material and a core material encapsulated in the wall material. The wall material comprises pectin and sodium carboxymethyl cellulose, and the core material comprises vitamin C, nitrate, and chitosan. The raw materials of the microcapsule comprise, in parts by weight: pectin 0.7-1.2 parts by weight, sodium carboxymethyl cellulose 0.7-1.2 parts by weight, chitosan 1 part by weight, and vitamin C and nitrate in a total amount of 0.3-1.2 parts by weight, the molar ratio of vitamin C to nitrate ions being 1:1-1:5. A use of the microcapsule in the preparation of a drug for preventing and/or treating damage to the salivary glands caused by radiation therapy, in particular a use in the preparation of a drug for preventing and/or treating damage to the salivary glands caused by radiation therapy for nasopharyngeal carcinoma.

### CROSS-REFERENCE TO RELATED APPLICATION

This application claims the benefit of Chinese Application No. 202110951722.5 filed on Aug. 19, 2021. The application No. 202110951722.5 is hereby incorporated by reference in its entirety.

### TECHNICAL FIELD

The invention belongs to the field of pharmaceutical preparation and medicine, and particularly relates to a microcapsule and the preparation method and application thereof.

### DESCRIPTION OF RELATED ART

Microencapsulation technology refers to the technology of embedding and encapsulating solid, liquid or gas in a tiny, closed vesicle to become a solid particle product. Generally, the encapsulated material is called the core material, and the encapsulating material is called the wall material. By microencapsulation, it is possible to protect a substance from the environment, to mask undesirable taste or odor, to modify the state, mass, or volume of a substance, to reduce toxicity, and to control release.

Combinations of vitamin C and nitrates have been shown to possess anti-tumor activity. However, vitamin C is very unstable. Environmental factors such as temperature, pH, oxygen, metal ions, ultraviolet radiation and X-ray can affect the stability of vitamin C. In addition, vitamin C and nitrates have good water solubility, and their half-life in vivo is only 2 hours after oral administration, so it is difficult to maintain effective blood concentration. These are the problems that are urgently demanded to be solved in the further development of compositions comprising vitamin C and nitrates.

Taking vitamin C as an example, microencapsulation of a water-soluble substance generally adopts a method, including phase separation by cooling, spray-drying, spray-cooling, freezing-melting, fluidized bed spraying, suspension coating, solvent evaporation, etc. The wall materials used are mainly selected from the group consisting of ethyl cellulose, carboxymethyl cellulose, gelatin, β-cyclodextrin, paraffin, etc. (Jing Legang et al., Preparation and applications of microencapsulation of vitamin C [J]. Chemical Industry and Engineering Progress, 2006, 25(11):1256-1259). However, how to improve the encapsulation efficiency of a water-soluble core material and reduce the degradation of a unstable substance (such as vitamin C) remains a challenge to those skilled in the art.

Due to the superficial anatomical position of the salivary gland, substantial damage tends to occur to the salivary gland by head and neck radiotherapy, resulting in a sharp decrease in salivary secretion function ([1] Jensen S B, et al. A systematic review of salivary gland hypofunction and xerostomia induced by cancer therapies: management strategies and economic impact. Supportive care in cancer: official journal of the Multinational Association of Supportive Care in Cancer. 2010; 18:1061-79. [2] Hu L, et al. Intragland Shh gene delivery mitigated irradiation-induced hyposalivation in a miniature pig model. Theranostics. 2018; 8:4321-31). Because of the reduced saliva secretion, patients feel dry in mouth, and have sensation of foreign body and burning. When chewing food, especially dry food, they can not form food balls and affect swallowing. Little saliva secretion leads to slight scouring effect on teeth and oral mucosa, deteriorating oral self-cleaning. In addition, the patient's sense of taste is also affected, the appetite can not be effectively stimulated, and the function of the entire digestive system will be affected. In a word, salivary gland damage caused by radiotherapy will result in reduced life quality of patients, which is an urgent problem in clinical practice. At present, however, there is no effective medication for salivary gland damage caused by radiotherapy in clinic.

### SUMMARY

In order to overcome the defects of the prior art, the invention provides a microcapsule of vitamin C and nitrate, a preparation method thereof and application thereof in preventing and treating salivary gland damage caused by radiotherapy.

A microcapsule of vitamin C and nitrate comprises a wall material, and a core material encapsulated in the wall material, wherein the wall material includes pectin and sodium carboxymethyl cellulose, and the core material includes vitamin C, nitrate and chitosan. The microcapsule comprises the following raw materials in parts by weight:

Pectin, 0.7-1.2 parts by weight; sodium carboxymethyl cellulose, 0.7-1.2 parts by weight; chitosan, 1 part by weight; and vitamin C and nitrate ions (in total), 0.3-1.2 part by weight, wherein the molar ratio of vitamin C to nitrate ranges from 1:1-1:5.

The microcapsule is prepared by the following method:
I. Preparing the raw materials based on their proportions;
II. Preparing the core material solution
   dissolving vitamin C, nitrate and chitosan in water to a final concentration of nitrate of about 4 mg/mL to obtain the core material solution;
III. Preparing the wall material solution
   uniformly mixing the wall material with water to a total mass percent concentration of the wall material in a range from 1.5 to 2.5% to obtain the wall material solution;
IV. Preparing the microcapsule
   uniformly mixing the core material solution prepared in step II and the wall material solution prepared in step III, freeze-drying, and crushing to obtain the microcapsules.

Preferably, the molar ratio of vitamin C to nitrate ions is from 1:1 to 1:4.

More preferably, the molar ratio of vitamin C to nitrate ions is 1:4.

Preferably, the nitrate is selected from the group consisting of sodium nitrate and/or potassium nitrate, and more preferably, the nitrate is sodium nitrate.

Preferably, the mass ratio of pectin, sodium carboxymethyl cellulose and chitosan is 0.85:0.85:1.

Preferably, the chitosan is chitosan 3000.

Preferably, the specific operation of the step II is as follows:
dissolving said parts by weight of vitamin C in water in a 20-25° C. water bath in dark, cooling to 2-4° C., adding said parts by weight of nitrate and chitosan, stirring to dissolve them to a final concentration of nitrate of about 4 mg/mL to obtain the core material solution.

Preferably, the specific operation of the step III is:
dissolving said parts by weight of sodium carboxymethyl cellulose in water at 70-80° C., and stirring or homogenizing under high pressure to obtain solution A; dissolving said parts by weight of pectin in water at 45-55° C. to obtain solution B, wherein the volume of the solution A is similar to that of the solution B; mixing the solution A and the solution B and stirring to homogeneity to a total mass percentage concentration of the wall material of 1.5%-2.5% in water, so as to obtain the wall material solution.

Preferably, the specific operation of the step IV is as follows:
mixing the core material solution prepared in the step II with the wall material solution prepared in the step III, rapidly dispersing for 20-40 s with a high-speed homogenizer at 10000 r/min, repeating for 3 times, and then stirring for 5-10 min with a magnetic stirrer at a speed of 800-1000 r/min; freezing the obtained mixed solution in a refrigerator at −80° C. for 12 to 24 hours, and freeze-drying the frozen solution in a vacuum freeze dryer for 12 to 24 hours; crushing the freeze-dried product, sieving the crushed product with a 100-mesh sieve, and collecting the undersize product to obtain the microcapsules, or crushing the freeze-dried product into powder by a superfine powder jet mill to obtain the microcapsules.

Preferably, the microcapsules have a particle size in a range from 850 to 1000 nm.

The invention also aims to provide a pharmaceutical composition, which comprises the microcapsule of vitamin C and nitrate, and pharmaceutically acceptable adjuvants.

The pharmaceutically acceptable adjuvants include, but not limited to: (1) diluent, such as starch, powdered sugar, dextrin, lactose, pregelatinized starch, microcrystalline fiber, inorganic calcium salt (such as calcium sulfate, calcium hydrogen phosphate, pharmaceutical calcium carbonate, etc.), mannitol, vegetable oil, polyethylene glycol, distilled water, etc.; (2) binder, such as distilled water, ethanol, starch slurry, sodium carboxymethyl cellulose, hydroxypropyl cellulose, methyl cellulose and ethyl cellulose, hydroxypropyl methylcellulose, etc; (3) disintegrant, such as dry starch, sodium carboxymethyl starch, low-substituted hydroxypropyl cellulose, crospolyvinylpyrrolidone, croscarmellose sodium, etc; (4) lubricant, such as magnesium stearate, aerosil, talc, hydrogenated vegetable oil, polyethylene glycols, magnesium lauryl sulfate, etc.

Preferably, the pharmaceutical composition is a clinically acceptable formulation.

More preferably, the pharmaceutical composition is an oral formulation.

Preferably, the oral formulation is selected from one of the following: a tablet, a capsule, a granule, a dry suspension, a suspension and an oral solution.

The invention also aims to provide the application of the microcapsule of vitamin C and nitrate or the pharmaceutical composition comprising the microcapsule of vitamin C and nitrate in the preparation of a medicament for preventing and/or treating salivary gland damage caused by radiotherapy.

As a preferred embodiment, the invention provides the application of the microcapsule of vitamin C and nitrate or the pharmaceutical composition comprising the microcapsule of vitamin C and nitrate in the preparation of a medicament for preventing and/or treating salivary gland damage caused by radiotherapy for nasopharyngeal carcinoma.

In the specification of the present application, “about” is intended to include values within the range of “the specified value±25%”, unless otherwise specified. If the final concentration of nitrate is about 4 mg/ml in the step of preparing the core material solution, the final concentration of nitrate in the core material solution may be 4±1 mg/ml, for example, any concentration in the range from 3 mg/ml to 5 mg/ml.

The microcapsule prepared by the preparation method of the invention has high embedding rate. The highest embedding rate (based on nitrate) was 87.2%, as determined by HPLC.

Sustained release dynamics has demonstrated that controlled release can be realized by the microcapsule provided by the invention in simulated intestinal and gastric juice. Pharmacodynamics has demonstrated that in the high-dose group, the salivary flow rate from the rat salivary gland can be improved to about 85% of that before radiotherapy by the microcapsule provided by the invention at 8 weeks after radiotherapy, and there is no significant difference of the AQP5 expression level regulated by the high-dose group relative to the control group. It has also demonstrated that the microcapsule of the invention can reverse the damage to the salivary gland by radiation. Animal experiments also demonstrate that via direct gastric gavage, the effect of nitrate alone or the mechanical mixture of vitamin C and nitrate on the reversion of the damage to the salivary gland by radiation is obviously inferior to that of the microcapsule of the invention. It is the microcapsule of the present invention that ensure the in vivo synergistic effect of the composition of vitamin C and nitrate.

### BRIEF DESCRIPTION OF DRA WINGS

The present invention will be further described with reference to the accompanying drawings.

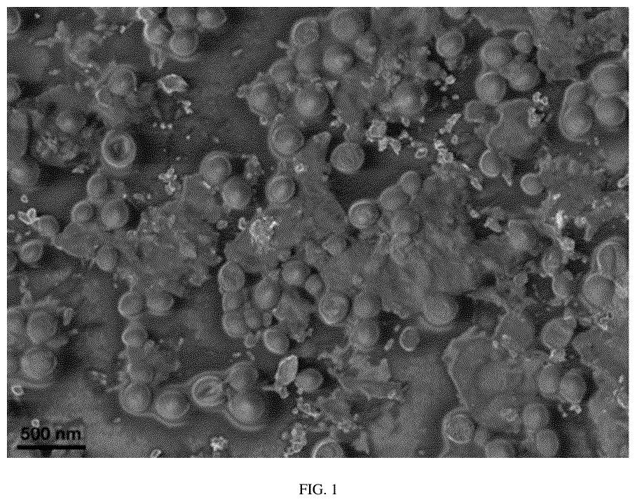

FIG. 1 shows a transmission electron microscopy photograph of the microcapsules prepared in Example 1 of the present invention.

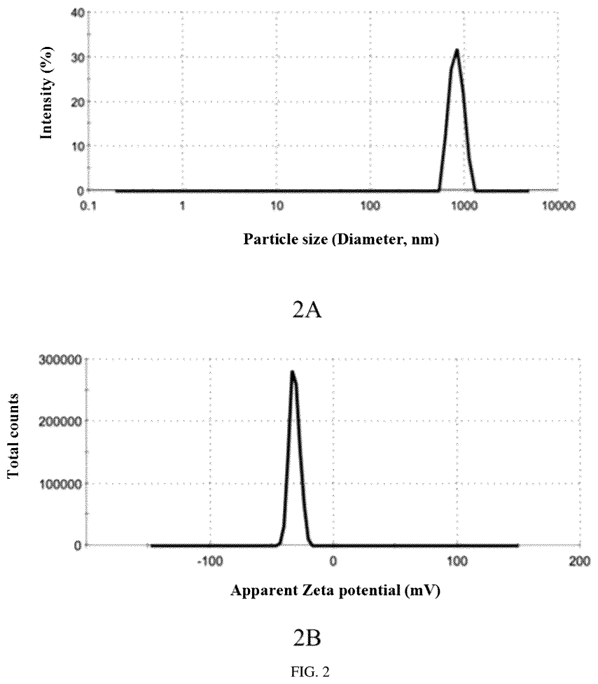

FIG. 2 shows the particle size distribution and the Zeta potential of the microcapsules prepared in Example 1 of the present invention, wherein FIG. 2A shows the particle size distribution and FIG. 2B shows the Zeta potential distribution.

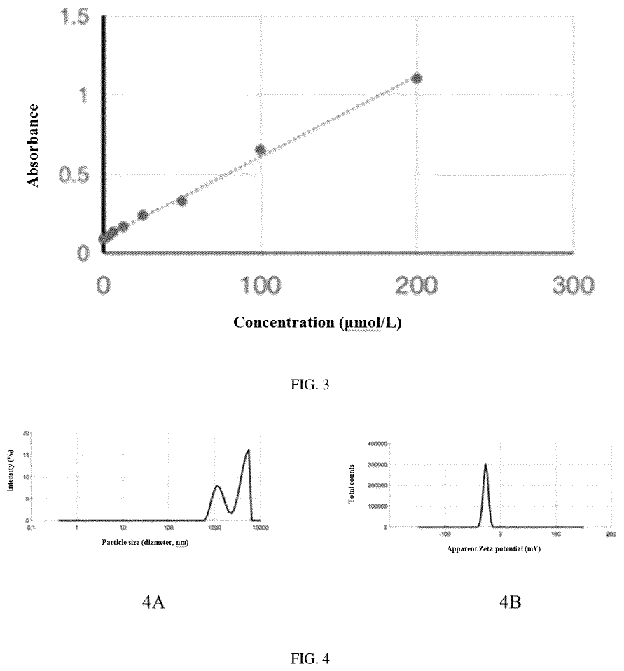

FIG. 3 shows a standard curve established for detection of the embedding rate of the microcapsules prepared in Example 1, wherein X axis represents the nitrate content and Y axis represents the absorbance value.

FIG. 4 shows the particle size distribution and the Zeta potential of the microcapsules prepared in Comparative Example 1, wherein FIGS. 4A, 4B and 4C shows the particle size distribution, the Zeta potential distribution, and the scanning electron microscopy photograph of the microcapsules, respectively.

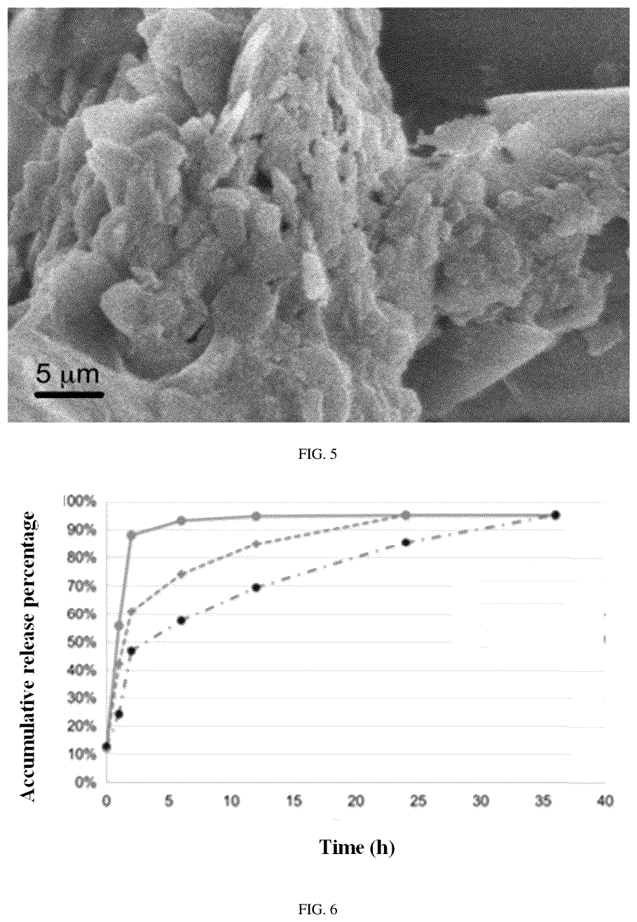

FIG. 5 shows a scanning electron microscopy photograph of the microcapsule prepared in Comparative Example 2.

FIG. 6 shows the cumulative release profile of the microcapsule prepared in Example 1 in simulated intestinal fluid and simulated gastric fluid as measured in Example 2, with time (h) on X axis and the cumulative release rate (%) on Y axis, in which:
[symbol] pH=7.2, [symbol] pH=6.5, [symbol] pH=2.0.

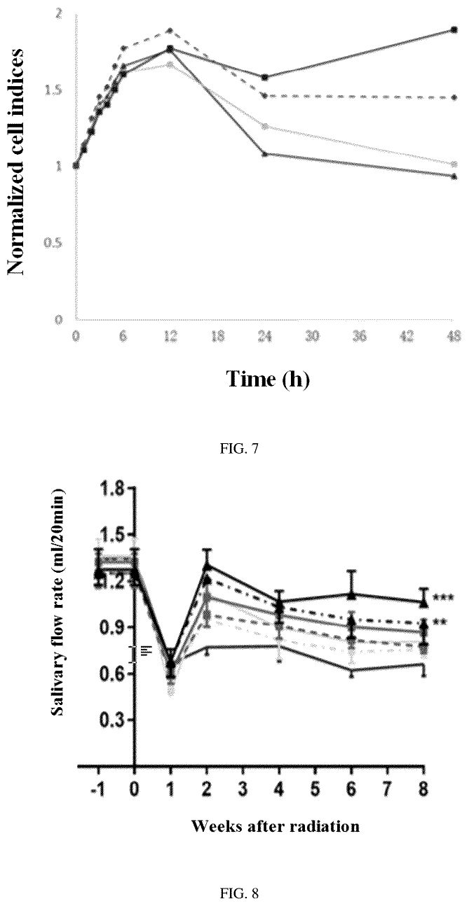

FIG. 7 shows the proliferation curve of submandibular gland epithelial cells after administrated with different drugs (sodium nitrate, microcapsule prepared in Example 1, and Vc) in Example 3, in which:

[symbol]

IR, [symbol] IR+Sodium nitrate, [symbol] IR+Microcapsules of Example 1, [symbol] IR+Vc

As shown in FIG. 7, the proliferation of submandibular gland epithelial cells can be improved by administration with different drugs, wherein the most significant improvement on the proliferation can be observed with the microcapsule of Example 1.

FIG. 8 shows the changes in the salivary flow rate of the rats in each group at 1 week before and at 1, 2, 4, 6 and 8 weeks after the radiotherapy in Example 4, in which:
[symbol] TH^{Group}, [symbol] TL^{Group}, [symbol] G0 H^{Group}, [symbol] G0 L^{Group},
[symbol] Nit H^{Group}, [symbol] Nit L^{Group}, [symbol] IR^{Group}.

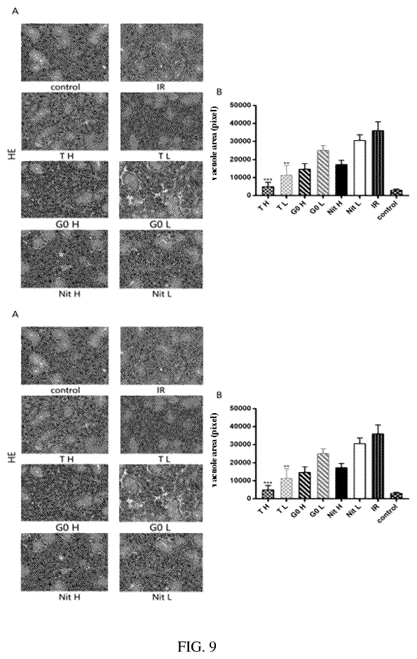

FIG. 9 is a microscopic photograph (×40) of the submandibular gland tissue after HR staining in each group (left side, A), and a quantitative analysis of the vacuole area in the submandibular gland acinus in each group (right side, B) at 8 weeks after radiotherapy in Example 4.

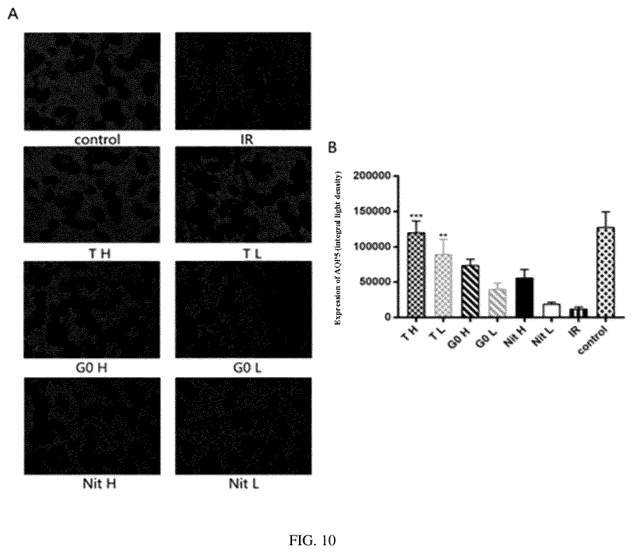

FIG. 10 shows the microscopic photograph (×20) of the paraffin section of AQP5 protein after immunofluorescence staining in each group (left side, A), and the quantification of AQP5 expression level in each group (right side, B) at 8 weeks after the radiotherapy in Example 4.

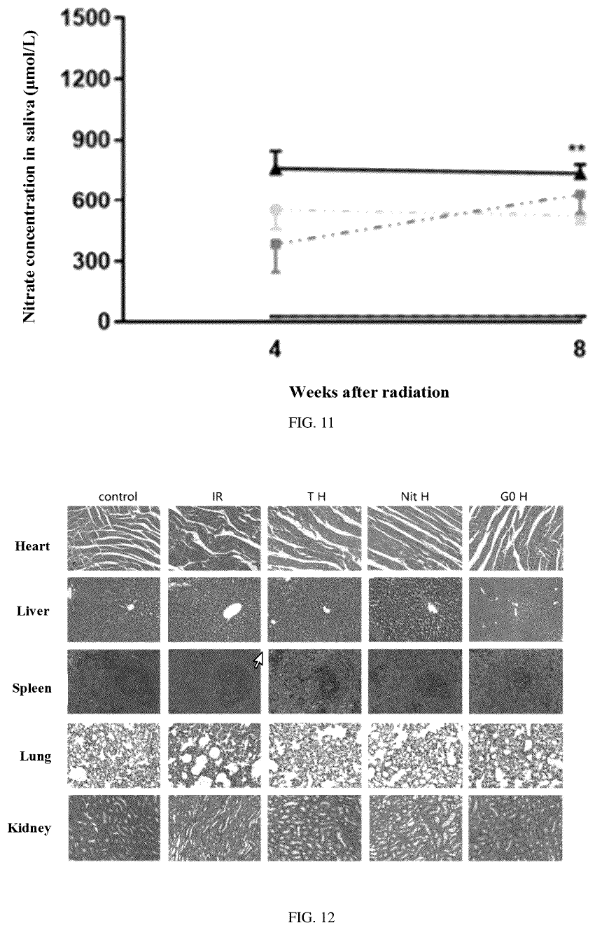

FIG. 11 shows the concentration of nitrate in the saliva of rats in each group at 4 weeks and 8 weeks after radiotherapy in Example 4, in which:
[symbol] TH^{Group}, [symbol] G0^{Group}, [symbol] Nit^{Group}, [symbol] IR^{Group}.

FIG. 12 is a microscopic photograph (×20) of the sections of major organs after HE staining in group control, IR, TH, Nit H, and G0 H, at 8 weeks after the radiotherapy in Example 4.

### DETAILED DESCRIPTION

The present invention will be described below with reference to specific Examples. It will be understood by those skilled in the art that these embodiments are merely illustrative of the invention and do not limit the scope of the invention in any way.

The experimental methods in the following Examples are all conventional methods, unless otherwise specified. The raw materials, reagents, and the like used in the following Examples are commercially available, unless otherwise specified.

### Example 1 Preparation of a Microcapsule of Vitamin C and Nitrate

### 1.1 Reagents and Instruments

Pectin, sodium carboxymethyl cellulose (CMC-Na), Nantong Tailida New Materials Co., Ltd.; chitosan (chitosan 3000, and chitosan 75000), Anhui Yuanzheng Bioengineering Co., Ltd.; vitamin C, sodium nitrate, Merck Ltd.

(Sigma-Aldrich); sodium heptanesulfonate, vitamin standard, Shanghai Yuanye Bio-Technology Co., Ltd; EDTA, isopropanol, triethylamine, glacial acetic acid, ethanol, Beijing Reagent Company.

Electronic balance BSA124, Sartorius Scientific Instruments Co., Ltd.; rotary evaporator LABOROTA4003, Heidolph Instruments GmbH & Co. KG (Germany); 101-0 electro-thermostatic blast oven, Tianjin Taisite Instrument Co., Ltd.; magnetic stirrer, ultrafine pulverizer, IKA Instrument Co., Ltd. (Germany); ultrafine jet mill, Model: R & D Jet Mills Lab; KQ-500DE digital control ultrasonic cleaner, Kunshan Ultrasonic Instruments Co., Ltd.; Vacuum freeze dryer Alpha1-4LDplus, MARTIN CHRIST corporation (Germany); Agilent 1200 HPLC, Agilent Technologies (U.S.).

### 1.2 Preparation of Microcapsules

### I. Preparation of the Raw Materials

14 mg of vitamin C, 27.5 mg of sodium nitrate, 95 mg of CMC-Na, 95 mg of pectin and 112 mg of chitosan (chitosan 3000) were weighed out.

### II. Preparation of the Core Material Solution

Vitamin C were placed in a flask, in to which 5 ml of pure water was added. The vitamin C was thoroughly dissolved in a water bath at 20-25° C. under dark, and then cooled to 4° C. in a refrigerator. Subsequently, sodium nitrate and chitosan were added and dissolved by stirring, so as to obtain a solution with a final nitrate concentration of 4 mg/mL, i.e., the core material solution.

### III. Preparation of the Wall Material Solution

CMC-Na was added into 5 ml pure water, heated in a water bath at 80° C. and stirred with a magnetic stirrer until it was completely dissolved to obtain solution A. Pectin was added into 4.7 ml of pure water, heated in a water bath at 50° C., and stirred until the pectin was completely dissolved to obtain solution B. The solution A and the solution B were mixed and stirred homogeneously to obtain the wall material solution, in which the total mass percentage concentration of the wall material in the water is 1.95%.

### IV. Preparation of the Microcapsules

The wall material solution and the core material solution were mixed and quickly dispersed by a high-speed homogenizer at a speed of 10000 r/min 30 s. The process was repeated for 3 times. The solution was then stirred by a magnetic stirrer to make it thoroughly mixed. The mixture was poured into a culture dish to a thickness of 2-5 cm, which was then placed in a refrigerator at −80° C. and frozen for 12-24 hours. The frozen solution was then freeze-dried in a vacuum freeze drier (Alpha1-4LDplus, MARTIN CHRIST corporation, Germany) for 12-24 hours. After freeze drying, the sample existed in flocculent structure. The flocculent sample was then pulverized into powder by a superfine jet mill to obtain the microcapsules. The resulting microcapsules were placed in a desiccator for later use.

### 1.3 Characterization and Related Tests of Microcapsules

### A. Morphological Observation

This product is light yellow powder. It was observed by a transmission electron microscope that the particle size was-890 nm with a relatively regular spheroid. The transmission electron microscopy photograph was shown in FIG. 1.

### B. Determination of Particle Size and Zeta Potential

The particle size of the microcapsules prepared in Example 1 was measured using a laser nanoparticle size analyzer, and the Zeta potential of the microcapsules was measured using a Zeta potential analyzer. The results were each shown in FIG. 2. As shown in FIG. 2A, the particle size of the microcapsules is normally distributed with the average particle size of 890.2±180.4 nm; while in FIG. 2B, the Zeta potential of the microcapsules was −31.5±4.2 mV.

### C. Determination of Embedding Rate

Since NO3- is the main active component, it was taken as the main analysis object. The overall embedding effect of the microcapsules is reflected by the content and embedding rate of nitrate in the microcapsules.

### C-1. Determination of the Content of Nitrate not Embedded in the Surface Layer of the Microcapsules:

30 mg of the microcapsules were placed into a beaker under dark, and washed by 15 mL 50% ethanol aqueous solution for 3 times. The supernatant was filtered, and the filtrates were combined. The combined filtrate was then centrifuged for 5 min at 3500 r/min and at 4° C. The supernatant after centrifugation was rotary evaporated under vacuum to dryness. Finally, the dried material was redissolved with 1 mL of pure water, and passed through a 0.45 μm organic filter membrane. The filtrate was then placed in a brown vial and stored at 4° C. for later use.

The nitrate content was determined using a total nitric oxide and nitrate/nitrite assay kit (PKGE001, R & D Systems, USA):

### Preparation:

1) The sample was placed at 4° C. for 2 h and centrifuged at 14000 rpm for 10 min, and the supernatant was pipetted out.

2) The resulting supernatant was filtered and 10-fold diluted to obtain reaction solution 1.

3) Preparation of reaction solution 2: the reaction solution 1 was 10-fold diluted using distilled water/deionized water to obtain reaction solution 2.

4) Preparation of nitrate reductase: the nitrate reductase solution was prepared with 1.0 mL of nitrate reductase stock solution, was vortexed strongly, stood at room temperature for 15 min, was vortexed and then stood at room temperature for another 15 min, was vortexed again and used immediately.

The nitrate reductase solution was diluted with the reaction solution 2 to prepare a nitrate reductase solution with the concentration of ⅕ of the stock solution. The steps were as follows:
a. Nitrate reductase (×well+2)×5 μL;
b. The reaction solution 2 was taken out with a volume equal to 4 folds of that of the solution used in the step a;
c. The solutions of steps a and b were added to a clean test tube, and vortexed;
d. It was placed on ice and used within 15 min.

5) NADH reagent—NADH was prepared with 5.0 mL of deionized or distilled water. The solution was allowed to stand for 3 minutes with gentle stirring before use. The solution should be used within 15 minutes or placed on ice.

6) Preparation of nitrate standard:

900 μL of the reaction solution 2 was pipetted into a 200 μmol/L tube, and a series of nitrate standards at the following concentrations were prepared: 100 μmol/L, 50 μmol/L, 25 μmol/L, 12.5 μmol/L, 6.25 μmol/L, and 3.12 μmol/L. Reaction solution 2 was used as blank (0 μmol/L).

### Steps for Detecting Nitrate Content (According to the Instruction of the Kit):

1) All reagents, standards, samples, and so on were prepared according to the previous preparation steps;
2) 100 μL of reaction solution 2 was added to the wells of the blank group;
3) 100 μL of the nitrate standard or the sample was each added to the remaining wells;
4) 50 μL NADH was added to all wells;
5) 50 μL of the diluted nitrate reductase was added to all wells, mixed well, and covered with tape;
6) Incubation at 37° C. for 30 min;
7) 100 μL of Griess I reaction solution was added to all wells;
8) 100 μL of Griess II reaction solution was added to all wells, sides of the plate were gently tapped, and the plate was mixed well;
9) Incubation at room temperature for 10 min;
10) Optical density (O.D.) was determined at a wavelength of 540 nm with a wavelength calibration of 690 nm;
11) A standard curve was established according to the measured values of the standards, and the contents of the nitrate in the samples in each group were calculated. The standard curve is shown in FIG. 3, and the regression equation is:

y=0.0051x+0.0985(R2=0.9965)

The result of the nitrate content on the surface of the microcapsules (not embedded) was shown in Table 1, where “n=6” indicated that 6 parallel tests were performed.

TABLE 1
Surface nitrate content of the microcapsules prepared in Example 1

| | Average value | SD |
|---|---|---|
| | (μmol/L) | (μmol/L) |
| nitrate content | 280.1 | 21.7 |

Note:
n = 6

### C-2. Assay of the Total Nitrate Content in the Microcapsules:

30 mg of the microcapsules were placed into a beaker under dark, into which 10 mL of pure water was added. The microcapsules were dissolved with the aid of ultrasound for 3 times, and the suspension was centrifuged at 3500 r/min and at 4° C. for 5 min. A certain amount of the supernatant was pipetted from it. The same determination method as above was performed. The result was shown in Table 2, where “n=6” indicated that 6 parallel tests were performed.

TABLE 2
Total nitrate content in the microcapsules prepared in Example 1

| | Average value | SD |
|---|---|---|
| | (μmol/L) | (μmol/L) |
| nitrate content | 2188.5 | 35.7 |

Note:
n = 6

### C-3. Calculation of Embedding Rate of the Microcapsules

The results obtained in C-1 and C-2 were substituted into the following equation:

Embedding Rate=(1−the content of nitrate not embedded in the surface layer of microcapsules/the total content of nitrate in microcapsules)×100%

After calculation, the embedding rate was 87.2% (n=6).

### Comparative Example 1. Preparation of Microcapsules of Vitamin C and Nitrate

The microcapsules of vitamin C and nitrate were prepared in this Comparative Example, in which the raw materials and methods were substantially the same as those in Example 1, except that the chitosan was chitosan 75000.

The particle size of the prepared microcapsules was measured by a laser nanoparticle analyzer, the Zeta potential of the microcapsules was measured by a Zeta potential analyzer, and the morphology of the microcapsules was observed by a scanning electron microscope. The results were shown in FIG. 4. In FIG. 4A, the particle size of the microcapsules prepared in this Comparative Example is not normally distributed, indicating that the size was not uniform. The particle size was close to 4408±943.1 nm. In FIG. 4B, the surface Zeta potential of the microcapsules prepared in this Comparative Example was reduced to −26.6 mV. In FIG. 4C, the microcapsules prepared in the present Comparative Example were irregular. Obviously, as compared with the microcapsules of Example 1, the particle size of the microcapsules of this Comparative Example has increased significantly, reaching the micron level, suggesting that it may be trapped by liver and is not suitable for sustained release. The absolute value of Zeta potential of the microcapsules in Comparative Example 1 was less than 30 mV, suggesting that the microcapsules tended to aggregate after dispersion with poor system stability.

### Comparative Example 2. Preparation of the Microcapsules of Vitamin C and Nitrate

The microcapsules were also prepared in this Comparative Example with the following method, in which the wall material was composed of CMC-Na and pectin, and the core material was composed of vitamin C and sodium nitrate:

### I. Preparation of the Raw Materials

14 mg of vitamin C, 27.5 mg of sodium nitrate, 95 mg of CMC-Na and 95 mg of pectin were weighed out.

### II. Preparation of the Core Material Solution

Vitamin C was added into a flask, into which 5 ml of pure water was added. Vitamin C was thoroughly dissolved in a 20-25° C. water bath under dark and cooled to 4° C. in a refrigerator. Subsequently, sodium nitrate was added, and dissolved by stirring to obtain the core material solution with a final nitrate concentration of 4 mg/mL.

### III. Preparation of the Wall Material Solution

CMC-Na was added into 5 ml pure water, heated in a water bath at 80° C. and stirred with a magnetic stirrer until it was completely dissolved to obtain solution A. Pectin was added into 4.7 ml of pure water, heated in a water bath at 50° C., and stirred until the pectin was completely dissolved to obtain solution B. The solution A and the solution B were mixed and stirred homogeneously to obtain the wall material solution, in which the total mass percentage concentration of the wall material in the water is 1.95%.

### IV. Preparation of the Microcapsules

The wall material solution and the core material solution were mixed and quickly dispersed by a high-speed homogenizer at a speed of 10000 r/min 30 s. The process was repeated for 3 times. The solution was then stirred by a magnetic stirrer to make it thoroughly mixed. The mixture was poured into a culture dish to a thickness of 2-5 cm, which was then placed in a refrigerator at −80° C. and frozen for 12-24 hours. The frozen solution was then freeze-dried in a vacuum freeze drier (Alpha1-4LDplus, MARTIN CHRIST corporation, Germany) for 12-24 hours. After freeze drying, the sample existed in flocculent structure. The flocculent sample was then pulverized into powder by a superfine jet mill to obtain the microcapsules. The resulting microcapsules were placed in a desiccator for later use.

The morphology of the prepared microcapsules was observed by a scanning electron microscopy. The results were shown in FIG. 5. As shown in FIG. 5, the microcapsules had many sheet-like structures and were not uniform in size, and there were almost no spherical particles.

### Example 2. Pharmacokinetic Test of the Microcapsules of Example 1

The sustained release kinetics of the microcapsules prepared in Example 1 was studied in this Example.

### 1. In Vitro Test

Preparation of simulated gastric juice: 3.2 g of pepsin and 2 g of sodium chloride were added to 7 mL of hydrochloric acid, and water was then added to a total volume of 1000 mL. The pH value of the solution was 1.2.

Preparation of simulated intestinal fluid: 6.8 g of potassium dihydrogen phosphate was dissolved in 250 ml of water, into which 77 mL of 0.2 mL/L sodium hydroxide solution and 500 mL of water were added. Subsequently, 10 g of trypsin was added. After dissolution, the pH was adjusted to 6.8 with sodium hydroxide solution or 0.2 mol/L hydrochloric acid solution, and then it was diluted to 1000 mL with water.

Methods: 50 mg of the microcapsules were accurately weighed out and put into a reagent bottle, after which 100 mL of simulated intestinal juice or simulated gastric juice was added. The reagent bottle was placed on a magnetic stirrer and stirred at a speed of 100 r/min. 0.5 mL of the supernatant sample was collected every 3 minutes, and diluted 10 times, so as to determine the content of nitrate therein. After collection, the release rate of nitrate was determined by immediately supplementing 0.5 mL of simulated intestinal fluid or simulated gastric fluid, and the final release of nitrate from the microcapsules in simulated intestinal fluid and simulated gastric fluid was investigated.

The results were shown in FIG. 6. As shown in FIG. 6, the microcapsules were relatively stable in neutral solution, and in simulated intestinal fluid and simulated gastric fluid. The degree of sustained release of nitrate in the microcapsules was significantly enhanced, and was maintained stable.

### 2. In Vivo Test

A total of 40 ICR mice (male, 20±4 g) were randomly divided into 4 groups of 10 mice each. Sodium nitrate, the microcapsules of Comparative Example 1, the microcapsules of Comparative Example 2, and the microcapsules of Example 1 were administered once at a dose of 2 mmol/kg sodium nitrate by gastric gavage. Blood was collected from vena caudalis at 0 h, and 2, 4, 6, 12, and 24 h after gastric gavage, respectively.

Nitrate detection method: the method was same as Example 1, except that the sample was the serum obtained from the tail vein blood of mouse after centrifugation. The results were shown in Table 3.

TABLE 3
Nitrate content in the blood of the mice
after single administration (μmol/L)

| Time | | Comparative | Comparative | |
|---|---|---|---|---|
| (h) | NaNO₃ | example 1 | example 2 | Example 1 |
| 0 | 41.8 | 42.2 | 43.1 | 42.7 |
| 2 | 1052.8 | 971.4 | 912.3 | 799.6 |
| 4 | 572.4 | 702.8 | 662.6 | 833.9 |
| 6 | 297.4 | 475.0 | 557.2 | 883.1 |
| 12 | 45.3 | 165.9 | 272.9 | 402.4 |
| 24 | 32.7 | 30.9 | 47.3 | 95.26 |

The data in Table 3 showed that the microcapsules of the present invention could achieve a sustained release and maintain an effective plasma concentration of nitrate for 6 to 12 hours as compared to the microcapsules prepared in Comparative Examples 1 and 2.

### Example 3. In Vitro Cell Experiments on the Microcapsules of the Invention

In this example, the protective effect of the microcapsules of the present invention (prepared according to the method described in Example 1) on human submandibular gland epithelial cells after irradiation was investigated by testing the proliferation of human submandibular gland epithelial cell line (HSG) (purchased from the ATCC Cell Bank) loaded with different drugs after irradiation.

### 2.1 Effect on Cell Proliferation:

1) Cells were plated into RTCA (RTCA S16, Agilent) cell proliferation assay plate at a density of 1×10⁴ cells per well. 4 hours after plating, the cells were adherent and in logarithmic growth phase. 10 μL of different drugs (sodium nitrate, 0.5 mM; the microcapsules of Example 1, 0.25 mM; VC, 60.325 μM; and normal saline in the control group) were added, and the changes of cell proliferation were continuously detected. The results were shown in Table 4.

2) The proliferation plate were taken out, and the medium was refreshed. Subsequently, 10 μL of drugs (sodium nitrate, 0.5 mM; the microcapsules of Example 1, 0.25 mM; VC, 60.325 μM, and normal saline in the control group) were added. The cells were subjected to single X-ray (RAD SOURCE 2000) irradiation at a dose of 1.22 gy/min and a total dose of 2 gy.

3) After irradiation, detection was continuously carried out. The medium was refreshed on the next day and 10 μL of drugs (sodium nitrate, 0.5 mM; the microcapsules of Example 1, 0.25 mM; VC, 60.325 μM; normal saline in the control group) were added. The results were shown in Table 5 and FIG. 7.

4) Data analysis.

TABLE 4
Changes of proliferation of the cells from the epithelial cell line
of human submandibular gland after the addition of different drugs

| Time | | Sodium | Microcapsules | |
|---|---|---|---|---|
| (hours) | Control | nitrate | of Example 1 | Vc |
| 0 | 1 | 1 | 1 | 1 |
| 1 | 0.93 ± 0.01 | 1.01 ± 0.01^{b} | 0.97 ± 0.00 | 0.99 ± 0.00 |
| 2 | 1.07 ± 0.00 | 1.16 ± 0.00^{b} | 1.12 ± 0.01 | 1.12 ± 0.00 |
| 3 | 1.25 ± 0.01 | 1.36 ± 0.02^{b} | 1.33 ± 0.02 | 1.31 ± 0.02 |
| 4 | 1.46 ± 0.02 | 1.57 ± 0.02^{b} | 1.58 ± 0.03 | 1.53 ± 0.03 |
| 5 | 1.65 ± 0.01 | 1.79 ± 0.02^{b} | 1.82 ± 0.03 | 1.76 ± 0.02 |
| 6 | 1.83 ± 0.01 | 1.98 ± 0.03^{b} | 2.05 ± 0.03 | 1.96 ± 0.02 |
| 7 | 1.99 ± 0.01 | 2.16 ± 0.02^{b} | 2.24 ± 0.02^{a} | 2.15 ± 0.04 |
| 8 | 2.13 ± 0.01 | 2.34 ± 0.04^{b} | 2.41 ± 0.04^{a} | 2.30 ± 0.01 |
| 9 | 2.26 ± 0.01 | 2.47 ± 0.06^{b} | 2.57 ± 0.06^{a} | 2.46 ± 0.02 |
| 10 | 2.40 ± 0.03 | 2.61 ± 0.05^{b} | 2.73 ± 0.03^{a} | 2.58 ± 0.03 |
| 11 | 2.50 ± 0.02 | 2.75 ± 0.06^{b} | 2.87 ± 0.03^{a} | 2.71 ± 0.03 |
| 12 | 2.63 ± 0.02 | 2.87 ± 0.06^{b} | 2.99 ± 0.05^{a} | 2.85 ± 0.01 |
| 13 | 2.71 ± 0.00 | 3.00 ± 0.07^{b} | 3.11 ± 0.11^{a} | 2.95 ± 0.03 |
| 14 | 2.79 ± 0.01 | 3.07 ± 0.06^{b} | 3.21 ± 0.11 | 3.07 ± 0.03 |
| 15 | 2.89 ± 0.01 | 3.18 ± 0.06^{b} | 3.32 ± 0.09^{a} | 3.16 ± 0.04 |
| 16 | 2.97 ± 0.05 | 3.30 ± 0.08^{b} | 3.45 ± 0.06^{a} | 3.27 ± 0.04 |

^{a}Significant difference as compared with the control group and the Vc group, p < 0.05;
^{b}Significant difference as compared with the control group, p < 0.05, No significant difference as compared with the VC group, p > 0.05.

The data in Table 4 showed that the microcapsules of Example 1 (0.25 mM) and nitrate (0.5 mM) could promote the proliferation of submandibular gland epithelial cells under normal conditions.

TABLE 5
Proliferation changes of human submandibular gland epithelial
cells after single irradiation with different drugs

| | | | IR + | |
|---|---|---|---|---|
| Time | | IR + sodium | microcapsule | |
| (hours) | IR | nitrate | of example 1 | IR + Vc |
| 0 | 1 | 1 | 1 | 1 |
| 1 | 1.105 ± 0.04 | 1.1041 ± 0.0005 | 1.1085 ± 0.009 | 1.1413 ± 0.0206 |
| 2 | 1.2416 ± 0.08 | 1.23 ± 0.006 | 1.2245 ± 0.0003 | 1.3124 ± 0.0605 |
| 3 | 1.4055 ± 0.12 | 1.3822 ± 0.0005 | 1.3571 ± 0.008 | 1.4577 ± 0.0898 |
| 4 | 1.449 ± 0.15 | 1.4204 ± 0.0031 | 1.4047 ± 0.0111 | 1.5195 ± 0.0941 |
| 5 | 1.5506 ± 0.16 | 1.4988 ± 0.0520 | 1.5009 ± 0.004 | 1.6577 ± 0.11363 |
| 6 | 1.6572 ± 0.18 | 1.6094 ± 0.07141 | 1.6028 ± 0.001 | 1.7707 ± 0.1553 |
| 12 | 1.7633 ± 0.24 | 1.6704 ± 0.1140 | 1.7727 ± 0.024 | 1.8884 ± 0.2043 |
| 24 | 1.0809 ± 0.10 | 1.2648 ± 0.0044^{a} | 1.5797 ± 0.0509^{b} | 1.4604 ± 0.1735 |
| 28 | 0.9357 ± 0.18 | 1.0113 ± 0.1354 | 1.8937 ± 0.0545^{c} | 1.4511 ± 0.0659^{b} |

Note:
^{a}significant difference as compared with IR group, p < 0.05;
^{b}significant difference as compared with IR group and IR + sodium nitrate group, p < 0.01;
^{c}significant difference as compared with IR group, IR + sodium nitrate group and IR + Vc group, p < 0.01.

Table 5 and FIG. 7 showed that the proliferation of submandibular gland epithelial cells was significantly inhibited after single irradiation, indicating that the radiation caused damage to the submandibular gland epithelial cells. The addition of sodium nitrate, Vc and the microcapsules of the present invention can all promote the proliferation of submandibular gland epithelial cells, but the microcapsules of Example 1 of the present invention had the most significant effect, which was stronger than sodium nitrate and Vc, respectively (p<0.01).

### Example 4. Study on the Microcapsules of the Invention for Prevention of Radiation Damage To Rat Salivary Gland

### 4.1 Experimental Animals:

male SD rats, 12-week old, weighing 490 g-510 g/rat, were purchased from SPF (Beijing) Biotechnology Co., Ltd. There were totally 60 rats with 10 rats in each group. Ethical approval No.: AEEI-2021-025, Department of Zoology, Capital Medical University.

### 4.2 Experiment Reagents:

Microcapsules (hereinafter referred to as “microcapsules”) prepared as described in Example 1 were prepared into 45 mM and 20.25 mM solutions with pure water, respectively;
Sodium nitrate, 40.5 mM and 20.25 mM solutions, prepared in pure water;
Compositions of sodium nitrate and vitamin C (molar ratio of 4:1) were mechanically mixed, and solutions containing 40.5 mM and 20.25 mM of nitrate were prepared with pure water, respectively.

### 4.3 Experimental Groups and Treatments:

The rats were randomly divided into 8 groups, n=10. These groups were as follows: (1) Radiotherapy alone (IR); (3) Low dose of microcapsules 20.25 mM in drinking water (T L); (4) High dose of microcapsules 40.5 mM in drinking water (T H); (5) Low dose of sodium nitrate 20.25 mM (Nit L); (6) High dose of sodium nitrate 40.5 mM in drinking water (Nit H); (7) Low dose of physical mixture of sodium nitrate and Vc (NaNO₃ 20.25 mM: Vc 5.06 mM, G0L); (8) High dose of physical mixture of sodium nitrate and Vc (NaNO₃ 40.5 mM: Vc 10.1 mM, G0H).

Based on observation, daily water intake of rats was 51.78±3.64 ml·day, so that the pharmaceutical dosage intake was about 4.5 mmol/kg·day in Nit L, Nit H, G0L, G0H and T H groups, and 2.25 mmol/kg·day in T L group.

The rats in TH, TL, Nit H, Nit L, G0L and G0H groups were continuously given drinking water with the corresponding concentration of drugs in it from 1 week before radiotherapy to 8 weeks after radiotherapy. IR group and control group were given common drinking water. All animals were free to eat.

### 4.4 Method for Radiotherapy:

Five rats were put in one group, fixed properly after anaesthesia, and laid on supine position in an accelerator therapy bed. The submandibular gland of each rat was covered with a 3.0 cm×3.0 cm parafilm with a thickness of 1 cm, which was a water-equivalent material. The total length of the submandibular glands from 5 rats was about 30 cm.

Irradiation mode of 2D source to skin distance was used. The irradiation source was 21EX linear accelerator from Varian Medical Systems, Inc. (USA). The average energy of radiation was 6 MV, the dose rate was 300 cGy/min, the irradiation field was 34 cm×3.0 cm, and the distance between the radiation source and the upper surface of the parafilm was 100 cm. According to EQD2 equation, a single dose of 15 Gy is equivalent to a total biological effect caused by a total dose of 31.25 Gy given for 16 times with 2 Gy every time. The center of the submandibular gland in the middle of the five rats was used as the center of radiation, and the 0° field was used for radiation.

### 4.5 Research Indices:

4.5.1 The rats were injected with pilocarpine nitrate at 1 week before radiotherapy, and at 1, 2, 4, 6 and 8 weeks after radiotherapy, and the whole saliva was collected 20 minutes after stimulation to detect the salivary flow rate.

4.5.2 The experimental animals were sacrificed at 8 weeks after radiotherapy, and the serum was collected for biochemical detection.

4.5.3 The submandibular gland and the major organs of the whole body (heart, liver, spleen, lung and kidney) were collected for histological examination.

### 5. Research Methods:

5.1 Whole saliva collection from the rats: After proper anaesthesia, the rats were injected I.P. with pilocarpine nitrate in aseptic normal saline at a concentration of 0.4 mg/ml and a dosage of 0.1 mg/100 g b. w. After the injection, the rats were laid on a 20° inclined rat board on prone position, with the head slightly inclined downward, so that the head was in a slightly lower position. About 5 minutes after the injection, after the first drop of saliva from the mouth of the rat, one end of the capillary pipette was placed at the bottom of the mouth, and the other end was placed at the bottom of a 1.5 ml centrifugal tube. The wall of the tube clenched by both upper and lower incisors, and the saliva was collected for 20 minutes. The amount of saliva collected was calculated based weight.

5.2 Collection of serum: The neck of the rate was fixed, and the rat was sacrificed by cervical dislocation. The rat's chest was quickly opened, and about 3 ml of blood was collected from the heart to the biochemical detection tube. After standing for about 30 min and centrifuging at 3000 rpm for 15 min, the supernatant was collected for component analysis in a blood biochemical analyzer.

5.3 Collection of submandibular gland and major organs and tissues of the whole body: after the experimental animals were sacrificed, the animals were placed on supine position, disinfected, incised at the neck to completely dissect the submandibular gland. Subsequently, tissues and organs such as lung, liver, spleen, kidney, heart and so on were collected. The tissues were washed with PBS, cut into 0.5 cm×0.5 cm, and fixed in 4% paraformaldehyde (pH 7.2) at 4° C. for 24-48 h. After dehydration, wax impregnation and embedding, the tissues were sectioned at a thickness of 4 μm and baked, followed by hematoxylin and eosin (HE) staining and immunohistochemistry fluorescence staining. The remaining fresh samples were placed in a refrigerator at −80° C.

### 6. Statistical Analysis:

SPSS 25.0 was used for statistical analysis, and the data were expressed as means±SE. One-way ANOVA or paired t test was used to compare the differences between groups, and chi-square test was used to compare the rates. P<0.05 was considered statistically significant.

### 7. Experimental Results

7.1 The microcapsules of the present invention can better protect the mandibular gland and reverse the salivary gland damage caused by radiotherapy.

The results of salivary flow rate of the rats in each experimental group at different times were shown in Table 6 and FIG. 8.

TABLE 6
Results of the salivary flow rate of the rats in the groups before
and after radiotherapy (ml/20 min, mean ± SD)

| Group | 1 W | 1 W | 2 W | 4 W | 6 W | 8 W |
|---|---|---|---|---|---|---|
| Control | 1.38 ± 0.21 | 1.20 ± 0.25 | 1.12 ± 0.14 | 1.05 ± 0.17 | 0.98 ± 0.12 | 0.85 ± 0.11 |
| IR | 1.34 ± 0.25 | 0.66 ± 0.17 | 0.77 ± 0.10 | 0.76 ± 0.22 | 0.62 ± 0.09 | 0.66 ± 0.17 |
| TL | 1.25 ± 0.17 | 0.68 ± 0.23 | 1.22 ± 0.06^{a} | 1.03 ± 0.20^{a} | 0.95 ± 0.15^{a} | 0.93 ± 0.28 |
| TH | 1.27 ± 0.36 | 0.68 ± 0.18 | 1.30 ± 0.20^{a} | 1.07 ± 0.12^{a} | 1.12 ± 0.30^{a} | 1.06 ± 0.18^{b} |
| Nit L | 1.34 ± 0.26 | 0.60 ± 0.25 | 0.95 ± 0.01^{a} | 0.82 ± 0.21^{a} | 0.74 ± 0.12 | 0.76 ± 0.09 |
| Nit H | 1.37 ± 0.24 | 0.48 ± 0.25 | 1.12 ± 0.20^{a} | 0.91 ± 0.17^{a} | 0.80 ± 0.09^{a} | 0.81 ± 0.13 |
| G0 L | 1.25 ± 0.19 | 0.59 ± 0.03 | 0.98 ± 0.12^{a} | 0.82 ± 0.07^{a} | 0.82 ± 0.07^{a} | 0.77 ± 0.06 |
| G0 H | 1.32 ± 0.11 | 0.60 ± 0.09 | 0.10 ± 0.21^{a} | 0.90 ± 0.14^{a} | 0.90 ± 0.14^{a} | 0.87 ± 0.12 |

Note:
^{a}significant difference as compared with IR group, p < 0.05;
^{b}significant difference as compared with IR group, Nit H group, Nit L group, G0H group and G0L group, p < 0.01.

The main function of salivary gland is to secrete saliva. Based on the continuous observation from 1 week before radiotherapy to 8 weeks after radiotherapy, it were found that at 8 weeks after radiotherapy, the salivary flow rate of the rats in IR group decreased to about 50% of that before radiotherapy, while the salivary flow rate of the rats in the microcapsule group, G0 group and the sodium nitrate group was improved to varying extents as compared with IR group, and the improvement was significantly dose-dependent. The effect in T H group was the best, which, at 8 weeks after radiotherapy, could recover to about 85% of the salivary flow rate before radiotherapy, as shown in FIG. 8. Therefore, the microcapsules of the invention can better improve the salivary flow rate and the tissue structure of the salivary gland after radiotherapy as compared with sodium nitrate alone, and the physical mixture of sodium nitrate and Vc.

At 8 weeks after radiotherapy, the micrographs (×40) of the HE stained submandibular gland tissues of the rats in each experimental group were shown in FIG. 9A on the right of the panel of FIG. 9. The quantitative results of the vacuole area in the submandibular gland acinar cells were shown in Table 7. See FIG. 9B on the left of the panel of FIG. 9.

TABLE 7
The quantitative results of the vacuole area in the submandibular
gland acinar cells in each group at 8 weeks after radiotherapy

| Group | [symbol] area |
|---|---|
| control | [symbol] |
| IR | [symbol] |
| T L | [symbol] ** |
| T H | [symbol] *** |
| Nit L | [symbol] |
| Nit H | [symbol] |
| G0 L | [symbol] |
| G0 H | [symbol] |

**p < 0.01, as compared to IR group, Nit H group, Nit L group, G0 L group and G0 H group;
***p < 0.05, as compared to control group, and p < 0.01 as compared to IR group, T L group, Nit H group, Nit L group, G0 L group and G0 H group. [symbol]
indicates data missing or illegible when filed

A large number of vacuoles were found in the cytoplasm of submandibular gland acinar cells in IR group by HE staining of paraffin-embedded tissues. As compared with IR group, the vacuole area of TH 40.5 mM group (TH), TL 20.25 M group (TL) and Nit H 40.5 M group (Nit H) was significantly decreased in a dose-dependent manner. The microcapsule groups (T L and T H) were superior to the sodium nitrate groups (Nit L and Nit H), and the physical mixture of sodium nitrate and Vc groups (G0L and G0H). Again, the vacuole area of the high dose microcapsule group (TH group) was most similar to that of the blank control group (control) (see A and B in FIG. 9).

7.2 AQP5 staining confirmed that the secretion function of salivary gland was good after microcapsule administration.

The immunofluorescence staining photographs (×20 under microscope) of the paraffin sections of the submandibular gland of the rats in each experimental group at 8 weeks after radiotherapy, and the quantitation results of the expression levels of AQP5 in the submandibular gland acinar cells in each group were shown in FIG. 10 and Table 8.

TABLE 8
Quantitative results of the expression level (IOD) of
AQP5 in the submandibular gland acinar cells in each
group at 8 weeks after radiotherapy (mean ± SD, n = 10)

| Group | AQP5 IOD |
|---|---|
| control | [symbol] |
| IR | [symbol] |
| T L | [symbol] ** |
| T H | [symbol] *** |
| Nit L | [symbol] |
| Nit H | [symbol] |
| G0 L | [symbol] |
| G0 H | [symbol] |

**p < 0.01, as compared to IR group, Nit H group, Nit L group, G0 L group and G0 H group;
***p < 0.05, as compared to control group, and p < 0.01 as compared to IR group, T L group, Nit H group, Nit L group, G0 L group and G0 H group. [symbol]
indicates data missing or illegible when filed

Aquaporin 5 (AQP5) is involved in salivary secretion. Decreased expression of AQP5 leads to decreased salivary secretion and output, resulting in xerostomia. Through the immunofluorescence staining of AQP5 in the paraffin sections of each group, it was found that AQP5 was evenly distributed on the apical membrane of salivary gland acinar cells under normal conditions, and continuous arcs could be seen in the staining results. However, radiotherapy would reduce the expression of AQP5, so that its density was sharply reduced, and it was scattered on the cytomembrane. Microcapsules could maintain the level of AQP5 in submandibular gland after radiotherapy in a dose-dependent manner, and the trend was similar to that of the salivary flow rate in rats. The expression levels of AQP5 in T H group, T L group, G0 H group, G0 L group, Nit H group and Nit L group were significantly different from those in IR group, but there was no significant difference of the AQP5 expression level between T H group and the control group. See FIG. 10 for details.

### 7.3 Nitrate Content in Saliva

Saliva samples before administration, and at 4 and 8 weeks after radiotherapy were selected for nitrate content detection, and the results were shown in Table 9 and FIG. 11.

The results showed that the nitrate content in saliva of T group and Nit group was relatively stable after administration, while the nitrate content in saliva of G0 group changed greatly at 4 and 8 weeks after radiotherapy. At 8 weeks after radiotherapy, significant difference was observed in T group relative to IR group and Nit group. The results showed that addition of vitamin C could promote the effect of nitrate ions on the submandibular gland and lead to the transportation of nitrate. The microcapsules of the invention can stably promote the utilization of more nitrate ions by the salivary gland. However, the significant change of nitrate content in G0 group at 4 weeks and 8 weeks may be due to the unstable metabolism of the nitrate in the body.

TABLE 9
Comparison of the nitrate content in saliva of the rats at 4 and 8 weeks
after radiotherapy (μmol/L, mean ± SD, n = 10)

| Group | 4 W | 8 W |
|---|---|---|
| IR | [symbol] | [symbol] |
| Control | [symbol] | [symbol] |
| T H | [symbol] | [symbol] |
| Nit H | [symbol] | [symbol] |
| G0 H | [symbol] | [symbol] |

Note:
^{a}Significant difference as compared with IR, p < 0.01;
^{b}Significant difference as compared with IR, Nit H, p < 0.01;
^{c}Significant difference as compared with IR, Nit H and G0H, p < 0.01. [symbol]
indicates data missing or illegible when filed

7.4 Continuous administration of 40.5 mM of the microcapsules of Example 1 and sodium nitrate for 9 weeks had no significant effect on the organs in the body of the rats.

In order to evaluate the safety of the microcapsules of the present invention and sodium nitrate, the blood samples of the rats in each group were collected at 8 weeks after radiotherapy for serum biochemical detection, including the liver function, kidney function, serum biochemical electrolyte, protein, blood lipid, bile pigment and total carbon dioxide. The detection results were shown in Table 10 to Table 16, and all data of each group were within the normal physiological range of a rat. At the same time, the sections of the major organs of the rats in different experimental groups were stained with HE, and no abnormality was found in the organs of the rats in T H group, G0 H group and Nit H group, as shown in FIG. 12.

TABLE 10
Comparison of the serum biochemical indexes for the liver function of the SD
rats at 8 weeks after submandibular gland irradiation (mean ± SD, n = 10)

| Group | ALT(U/L) | AST(U/L) | ALP(U/L) | GGT(U/L) | LDH(U/L) |
|---|---|---|---|---|---|
| control | 56.50 ± 21.92 | [symbol] ± 28.28 | 136.00 ± 14.14 | 5.50 ± 0.71 | 946.30 ± 236.88 |
| IR | 56.25 ± 7.85 | 188.60 ± 14.61 | 135.20 ± 75.57 | 3.80 ± 0.84 | 1246.42 ± 354.90 |
| T L | 39.75 ± 14.61 | 94.25 ± 16.92 | 183.75 ± 91.18 | 4.25 ± 2.06 | 655.80 ± 133.66 |
| T H | 57.33 ± 13.31 | 148.00 ± 45.21 | 106.67 ± 27.93 | 6.67 ± 1.15 | 892.33 ± 413.55 |
| Nit L | 46.60 ± 16.80 | 123.80 ± 38.49 | 95.00 ± 52.85 | 5.60 ± 0.89 | 1212.44 ± 728.19 |
| Nit H | 49.20 ± 10.30 | 116.20 ± 9.44 | 112.00 ± 19.69 | 5.00 ± 0.71 | 747.40 ± 165.67 |
| G0 L | 42.75 ± 7.41 | 121.25 ± 25.85 | 118.00 ± 20.61 | 7.75 ± 0.50 | 950.73 ± 186.57 |
| G0 H | 43.33 ± 5.03 | 149.25 ± 16.10 | 97.50 ± 44.67 | 7.75 ± 0.96 | 1108.83 ± 259.1 |

ALP: alkaline phosphatase;
ALT: alanine aminotransferase;
AST: aspartate aminotransferase;
LDH: lactate dehydrogenase;
GGT: γ-glutamyltransferase [symbol]
indicates data missing or illegible when filed

TABLE 11
Comparison of the serum biochemical indexes for the
renal function of the SD rats at 8 weeks after submandibular
gland irradiation (mean ± SD, n = 10)

| Group | Cr (μmol/L) | UA (mmol/L) | Urea (mmol/L) |
|---|---|---|---|
| control | 51.35 ± 7.28 | 81.15 ± 20.57 | 5.76 ± 0.86 |
| IR | 34.64 ± 5.67 | 116.7 ± 24.3 | 5.54 ± 0.27 |
| T L | 35.2 ± 3.21 | 92.95 ± 30.27 | 5.73 ± 0.26 |
| T H | 47.86 ± [symbol] | 73.95 ± 7.23 | 6.26 ± 0.53 |
| Nit L | 47.82 ± 12.95 | 93.16 ± 18.63 | [symbol] ± 0.41 |
| Nit H | 46.7 ± 14.59 | 75.68 ± 8.33 | 5.78 ± 1.21 |
| G0 L | 39.3 ± 3.16 | 71.43 ± 10.46 | 6.71 ± 0.64 |
| G0 H | 61.00 ± 4.00 | 82.35 ± 12.31 | 6.58 ± 0.70 |

Cr: creatinine; UA: uric acid; Urea: urea. [symbol]
indicates data missing or illegible when filed

TABLE 12
Comparison of the serum biochemical indexes for the electrolyte of the SD
rats at 8 weeks after submandibular gland irradiation (mean ± SD, n = 10)

| | Ca | K | Cl | P | Na |
|---|---|---|---|---|---|
| Group | (mmol/L) | (mmol/L) | (mmol/L) | (mmol/L) | (mmol/L) |
| control | 2.53 ± 0.08 | 5.36 ± 0.17 | 103.10 ± 2.12 | 2.51 ± 0.39 | 137.95 ± 1.34 |
| IR | 2.35 ± 0.11 | 5.89 ± 0.47 | 104.00 ± 0.64 | 2.00 ± 0.50 | 138.58 ± 1.05 |
| T L | 2.39 ± 0.10 | 5.91 ± 0.55 | 103.32 ± 1.93 | 2.23 ± 0.67 | 138.15 ± 2.21 |
| T H | 2.40 ± 0.03 | 6.16 ± 0.92 | 150.43 ± 2.69 | 2.53 ± 0.56 | 139.36 ± 1.36 |
| Nit L | 2.39 ± 0.09 | 5.31 ± 0.48 | 104.54 ± 1.23 | 2.37 ± 0.22 | 140.72 ± 1.47 |
| Nit H | 2.41 ± 0.14 | 5.10 ± 0.45 | 104.16 ± 1.15 | 2.77 ± 1.01 | 141.66 ± 1.62 |
| G0 L | 2.47 ± 0.09 | 5.33 ± 0.37 | 105.90 ± 1.56 | 2.14 ± 0.15 | 140.58 ± 1.23 |
| G0 H | 2.29 ± 0.07 | 5.16 ± 0.09 | 105.55 ± 0.94 | 2.05 ± 0.30 | 141.23 ± 1.27 |

TABLE 13
Comparison of the serum biochemical indexes for
the proteins of the SD rats at 8 weeks after submandibular
gland irradiation (mean ± SD, n = 10)

| Group | TP (g/L) | Glb (g/L) | Alb (g/L) |
|---|---|---|---|
| control | 58.40 ± 6.36 | 48.70 ± 5.66 | 9.70 ± 0.71 |
| IR | 52.74 ± 4.50 | 43.20 ± 3.69 | 9.54 ± 1.35 |
| T L | 55.92 ± 6.74 | 46.60 ± 3.50 | 9.33 ± 3.71 |
| T H | 53.43 ± 4.74 | 44.53 ± 4.57 | 8.90 ± 0.20 |
| Nit L | 52.88 ± 2.40 | 44.68 ± 1.80 | 8.20 ± 1.32 |
| Nit H | 54.08 ± 4.11 | 45.42 ± 3.88 | 8.66 ± 0.87 |
| G0 L | 59.68 ± 2.29 | 49.18 ± 2.35 | 10.50 ± 0.60 |
| G0 H | 55.60 ± 2.08 | 45.68 ± 2.15 | 9.93 ± 0.47 |

TP: total protein;
Glb: globulin;
Alb: albumin

TABLE 14
Comparison of serum biochemical indexes for the lipids
of the SD rats at 8 weeks after submandibular gland irradiation
(mean ± SD, n = 10)

| | TG | TC | HDL-C | LDL-C |
|---|---|---|---|---|
| Group | (mmol/L) | (mmol/L) | (mmol/L) | (mmol/L) |
| control | 0.79 ± 0.23 | 1.85 ± 0.04 | 2.03 ± 0.08 | 0.20 ± 0.00 |
| IR | 0.56 ± 0.32 | 1.696 ± 0.40 | 1.76 ± 0.40 | 0.27 ± 0.10 |
| T L | 0.83 ± 0.29 | 1.73 ± 0.48 | 1.64 ± 0.72 | 0.33 ± 0.06 |
| T H | 0.29 ± 0.13 | 1.66 ± 0.08 | 1.79 ± 0.13 | 0.23 ± 0.03 |
| Nit L | 0.57 ± 0.42 | 2.05 ± 0.71 | 2.13 ± 0.71 | 0.27 ± 0.06 |
| Nit H | 0.31 ± 0.38 | 1.87 ± 0.38 | 2.09 ± 0.43 | 0.24 ± 0.06 |
| G0 L | 0.39 ± 0.33 | 2.11 ± 0.28 | 2.33 ± 0.37 | 0.35 ± 0.06 |
| G0 H | 0.50 ± 0.52 | 1.53 ± 0.46 | 1.75 ± 0.48 | 0.29 ± 0.08 |

TG: triglyceride;
TC: total cholesterol;
HDL-C: high-density lipoprotein cholesterol;
LDL-C: Low density lipoprotein cholesterol

TABLE 15
Comparison of the serum biochemical indexes for
the bilirubin of the SD rats at 8 weeks after submandibular
gland irradiation (mean ± SD, n = 10)

| Group | D-Bil(μmol/L) | T-Bil(μmol/L) | I-Bil(μmol/L) |
|---|---|---|---|
| control | 1.15 ± 0.49 | 1.45 ± 0.07 | 0.30 ± 0.56 |
| IR | 1.30 ± 0.84 | 3.83 ± 0.71 | 2.56 ± 0.32 |
| T L | 0.73 ± 0.15 | 2.08 ± 0.59 | 1.56 ± 0.55 |
| T H | 0.83 ± 0.21 | 2.37 ± 0.38 | 1.53 ± 0.31 |
| Nit L | 1.43 ± 0.40 | 2.52 ± 1.37 | 1.43 ± 1.07 |
| Nit H | 1.8 ± 1.21 | 3.52 ± 2.00 | 1.73 ± 1.57 |
| G0 L | 1.23 ± 0.17 | 2.85 ± 0.35 | 1.63 ± 0.39 |
| G0 H | 1.25 ± 0.21 | 2.23 ± 0.55 | 0.98 ± 0.59 |

D-Bil: direct bilirubin;
T-Bil: total bilirubin;
I-Bil: indirect Bilirubin

TABLE 16
Comparison of the serum biochemical indexes for total
carbon dioxide of the SD rats at 8 weeks after submandibular
gland irradiation (mean ± SD, n = 10)

| Group | TCO₂ (mmol/L) |
|---|---|
| control | 29.05 ± [symbol] |
| IR | 28.8 ± 1.25 |
| T L | [symbol] |
| T H | 30.43 ± [symbol] |
| Nit L | 30.26 ± 2.68 |
| Nit H | 30.12 ± 3.07 |
| G0 L | 26.38 ± [symbol] |
| G0 H | 26.03 ± [symbol] |

TCO₂: total carbon dioxide [symbol]
indicates data missing or illegible when filed

FIG. 12 showed that no abnormality was found in the organs of the rats in T H group, G0 H group, and Nit H group.

The results in Table 10 to Table 16 and FIG. 12 showed that the microcapsules of the present invention had no significant effect on the functions and physiological structures of various organs of the rats, and were safe for administration.

### Example 5. Microcapsules of Vitamin C and Nitrate (Molar Ratio of 1:1)

### I. Preparation of the Raw Materials

52.8 mg of vitamin C, 27.5 mg of sodium nitrate, 95 mg of CMC-Na, 95 mg of pectin, and 112 mg of chitosan (chitosan 3000) were weighed out.

### II. Preparation of the Core Material Solution

Vitamin C were placed in a flask, into which 5 ml of pure water was added. The vitamin C was thoroughly dissolved in a water bath at 20-25° C. under dark, and then cooled to 4° C. in a refrigerator. Subsequently, sodium nitrate and chitosan were added and dissolved by stirring, so as to obtain a solution with a final nitrate concentration of 4 mg/mL, i.e., the core material solution.

### III. Preparation of the Wall Material Solution

CMC-Na was added into 5 ml pure water, heated in a water bath at 80° C. and stirred with a magnetic stirrer until it was completely dissolved to obtain solution A. Pectin was added into 4.7 ml of pure water, heated in a water bath at 50° C., and stirred until the pectin was completely dissolved to obtain solution B. The solution A and the solution B were mixed and stirred homogeneously to obtain the wall material solution, in which the total mass percentage concentration of the wall material in the water is 1.95%.

### IV. Preparation of the Microcapsules

The wall material solution and the core material solution were mixed and quickly dispersed by a high-speed homogenizer at a speed of 10000 r/min 30 s. The process was repeated for 3 times. The solution was then stirred by a magnetic stirrer to make it thoroughly mixed. The mixture was poured into a culture dish to a thickness of 2-5 cm, which was then placed in a refrigerator at −80° C. and frozen for 12-24 hours. The frozen solution was then freeze-dried in a vacuum freeze drier (Alpha1-4LDplus, MARTIN CHRIST corporation, Germany) for 12-24 hours. After freeze drying, the sample existed in flocculent structure. The flocculent sample was then pulverized into powder by a superfine jet mill to obtain the microcapsules. The resulting microcapsules were placed in a desiccator for later use.

The microcapsules prepared in this Example were observed using an electron microscope, which showed a spherical shape with uniform size. The average particle size was measured to be 950±150.2 nm.

Microcapsules of Vitamin C and Nitrate (Molar Ratio of about 1:2)

### I. Preparation of the Raw Materials

26.4 mg of vitamin C, 27.5 mg of sodium nitrate, 95 mg of CMC-Na, 95 mg of pectin and 112 mg of chitosan (chitosan 3000) were weighed out.

### II. Preparation of the Core Material Solution

Vitamin C were placed in a flask, into which 5 ml of pure water was added. The vitamin C was thoroughly dissolved in a water bath at 20-25° C. under dark, and then cooled to 4° C. in a refrigerator. Subsequently, sodium nitrate and chitosan were added and dissolved by stirring, so as to obtain a solution with a final nitrate concentration of 4 mg/mL, i.e., the core material solution.

### III. Preparation of the Wall Material Solution

CMC-Na was added into 5 ml pure water, heated in a water bath at 80° C. and stirred with a magnetic stirrer until it was completely dissolved to obtain solution A. Pectin was added into 4.7 ml of pure water, heated in a water bath at 50° C., and stirred until the pectin was completely dissolved to obtain solution B. The solution A and the solution B were mixed and stirred homogeneously to obtain the wall material solution, in which the total mass percentage concentration of the wall material in the water is 1.95%.

### IV. Preparation of the Microcapsules

The wall material solution and the core material solution were mixed and quickly dispersed by a high-speed homogenizer at a speed of 10000 r/min 30 s. The process was repeated for 3 times. The solution was then stirred by a magnetic stirrer to make it thoroughly mixed. The mixture was poured into a culture dish to a thickness of 2-5 cm, which was then placed in a refrigerator at −80° C. and frozen for 12-24 hours. The frozen solution was then freeze-dried in a vacuum freeze drier (Alpha1-4LDplus, MARTIN CHRIST corporation, Germany) for 12-24 hours. After freeze drying, the sample existed in flocculent structure. The flocculent sample was then pulverized into powder by a superfine jet mill to obtain the microcapsules. The resulting microcapsules were placed in a desiccator for later use.

The microcapsules prepared in this Example were observed using an electron microscope, which showed a spherical shape with uniform size. The average particle size was measured to be 915±160.4 nm.

1. A microcapsule of vitamin C and nitrate, comprising a wall material and a core material encapsulated in the wall material, wherein the wall material comprises pectin and sodium carboxymethyl cellulose, and the core material comprises vitamin C, nitrate and chitosan;
wherein the microcapsule comprises the following raw materials in parts by weight:
pectin, 0.7-1.2 parts by weight; sodium carboxymethyl cellulose, 0.7-1.2 parts by weight; chitosan, 1 part by weight; and vitamin C and nitrate (in total), 0.3-1.2 parts by weight, wherein the molar ratio of vitamin C to nitrate ions ranges from 1:1 to 1:5; and
wherein the microcapsule is prepared by the following method:
I. preparing the raw materials based on their proportions;
II. preparing the core material solution by
dissolving vitamin C, nitrate and chitosan in water to a final concentration of nitrate of 4 mg/mL to obtain the core material solution;
III. preparing the wall material solution by
uniformly mixing the wall material with water to a total mass percent concentration of the wall material in a range from 1.5 to 2.5% to obtain the wall material solution; and
IV. preparing the microcapsule by
uniformly mixing the core material solution prepared in step II and the wall material solution prepared in step III, freeze-drying, and crushing to obtain the microcapsules.

2. The microcapsule according to claim 1, wherein the molar ratio of vitamin C to nitrate ions is in the range from 1:1 to 1:4.

3. The microcapsule according to claim 2, wherein the molar ratio of vitamin C to nitrate ions is 1:4.

4. The microcapsule according to claim 1, wherein the nitrate is selected from the group consisting of sodium nitrate and/or potassium nitrate.

5. The microcapsule according to claim 4, wherein the nitrate is sodium nitrate.

6. The microcapsule according to claim 1, wherein the mass ratio of pectin, sodium carboxymethyl cellulose, and chitosan is 0.85:0.85:1.

7. The microcapsule according to claim 1, wherein the chitosan is chitosan 3000.

8. The microcapsule according to claim 1, wherein the step II comprises:
dissolving said parts by weight of vitamin C in water in a 20-25° C. water bath in dark, cooling to 2-4° C., adding said parts by weight of nitrate and chitosan, stirring to dissolve them to a final concentration of nitrate of about 4 mg/mL to obtain the core material solution.

9. The microcapsule according to claim 1, wherein the step III comprises:
dissolving said parts by weight of sodium carboxymethyl cellulose in water at 70-80° C., and stirring or homogenizing under high pressure to obtain solution A; dissolving said parts by weight of pectin in water at 45-55° C. to obtain solution B, wherein the volume of the solution A is similar to that of the solution B; mixing the solution A and the solution B and stirring to homogeneity to a total mass percentage concentration of the wall material of 1.5%-2.5% in water, so as to obtain the wall material solution.

10. The microcapsule according to claim 1, wherein the step IV comprises:
mixing the core material solution prepared in the step II with the wall material solution prepared in the step III, rapidly dispersing for 20-40 s with a high-speed homogenizer at 10000 r/min, repeating for 3 times, and then stirring for 5-10 min with a magnetic stirrer at a speed of 800-1000 r/min; freezing the obtained mixed solution in a refrigerator at −80° C. for 12 to 24 hours, and freeze-drying the frozen solution in a vacuum freeze dryer for 12 to 24 hours; crushing the freeze-dried product, sieving the crushed product with a 100-mesh sieve, and collecting the undersize product to obtain the microcapsules, or crushing the freeze-dried product into powder by a superfine powder jet mill to obtain the microcapsules.

11. The microcapsule according to claim 1, wherein the microcapsules have a particle size of 850-1000 nm.

12. A pharmaceutical composition, comprising the microcapsule of vitamin C and nitrate according to claim 1, and pharmaceutically acceptable adjuvants.

13. The pharmaceutical composition according to claim 12, wherein the pharmaceutical composition is a clinically acceptable formulation.

14. The pharmaceutical composition according to claim 13, wherein the pharmaceutical composition is an oral formulation.

15. The pharmaceutical composition according to claim 14, wherein the oral formulation is the one selected from the group consisting of a tablet, a capsule, a granule, a dry suspension, a suspension, and an oral liquid.

16-17. (canceled)
